SEQUENCE LISTING (6 sequences)

SEQ ID NO: 1 - protein, 9 aa, Artificial Sequence
  Synthetic peptide
YPYDVPDYA

SEQ ID NO: 2 - protein, 8 aa, Artificial Sequence
  Synthetic peptide
DYKDDDDK

SEQ ID NO: 3 - DNA, 1808 bp, Artificial Sequence
  Synthetic polynucleotide
atgtggactctggtctcatgggtggctctgactgctggactggtggctggaaccgactacaaggacgacgacgacaaactcgctgccctgacggccagttctgcgacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctca

SEQ ID NO: 4 - protein, 601 aa, Artificial Sequence
  Synthetic peptide
MWTLVSWVALTAGLVAGTDYKDDDDKRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 5 - DNA, 1809 bp, Artificial Sequence
  Synthetic polynucleotide
atgtggactctggtctcatgggtggctctgactgctggactggtggctggaacctacccatacgatgttccagattacgctcgctgccctgacggccagttctgcgacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctca

SEQ ID NO: 6 - protein, 601 aa, Artificial Sequence
  Synthetic peptide
MWTLVSWVALTAGLVAGTYPYDVPDYARCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQL

(19) United States
(12) Patent Application Publication
(10) Pub. No.: US 2025/0264475 A1
(43) Pub. Date: Aug. 21, 2025

(54) METHODS FOR IDENTIFYING BINDING PARTNERS OF PROGRANULIN

(71) Applicant: Janssen Pharmaceutica NV, Beerse (BE)

(72) Inventors: Wei-zheng zeng, San Diego, CA (US); Joseph Chang, La Jolla, CA (US); Pascal Bonaventure, La Jolla, CA (US); Changlu Liu, San Diego, CA (US); David S Bredt, La Jolla, CA (US)

(21) Appl. No.: 18/288,025

(22) PCT Filed: Apr. 22, 2022
(86) PCT No.: PCT/EP2022/060724

§ 371 Date: Oct. 23, 2023

Related U.S. Application Data
(60) Provisional application No. 63/178,831, filed on Apr. 23, 2021.

(51) Int. Cl. G01N 33/68; G01N 1/30; G01N 21/64; G01N 33/58

(52) U.S. Cl. CPC G01N 33/68; G01N 1/30; G01N 21/6428; G01N 33/582; G01N 2021/6439

(57) ABSTRACT

Disclosed herein are screening methods for identifying cell surface receptors for progranulin. Also disclosed herein are screening methods for identifying intracellular proteins bind to progranulin.

### CROSS REFERENCE TO RELATED APPLICATION

This application claims the benefit of U.S. Provisional Application 63/178,831, filed on Apr. 23, 2021, which is incorporated by reference herein in its entirety.

### FIELD OF INVENTION

This invention relates to high-throughput screening methods for identifying binding partners of progranulin.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 28, 2022, is named PRD4129WOPCT1_SL.txt and is 16,384 bytes in size.

### BACKGROUND OF INVENTION

Neurodegenerative diseases occur when nervous system cells (neurons) in the brain and spinal cord begin to deteriorate. Today, 5.4 million Americans suffer from Alzheimer's disease; 500,000 from Parkinson's disease; and 50,000-60,000 from frontotemporal dementia (FTD). Because neurodegenerative diseases strike primarily in mid-to late-life, the incidence is expected to soar as the population ages. By 2030, as many as 1 in 5 Americans will be over the age of 65. Finding treatments and cures for neurodegenerative diseases is a goal of increasing urgency.

Progranulin (PGRN) is a cysteine-rich, secreted protein encoded by the GRN gene and known to be involved in neurodegeneration and other diseases. A reduction of PGRN levels in the brain correlates with familial forms of frontotemporal lobar degeneration (FTLD). Despite the strong correlation, its biological mechanism that causes neurodegeneration remains unclear. Though there have been proposed cell surface receptors that bind to PGRN, the link between those receptors and neurodegeneration remains murky, suggesting other binding partners may be at play. There is a need for methods to identify those interaction partners in a high-throughput and unbiased manner.

### BRIEF SUMMARY OF THE INVENTION

Provided herein is a high-throughput screening method for identifying transmembrane proteins that bind progranulin (PGRN), comprising: a) providing a cDNA collection comprising a plurality of expression vectors, each expression vector encoding a distinct transmembrane protein; b) providing a plate comprising a plurality of wells, wherein one or more wells thereof each contains host cells; c) into each of the one or more wells, introducing a distinct expression vector from said collection, thereby producing distinct transfected cells in each well; d) culturing the transfected cells under a condition to allow expression of a distinct transmembrane protein in each well; e) contacting the transfected cells in each of the one or more wells with a target protein comprising PGRN attached to a detectable tag; f) fixing the transfected cells with a fixing agent after contact with the target protein; g) washing the fixed cells; and h) after washing the fixed cells, determining the presence of the detectable tag in each of the one or more wells, wherein if the detectable tag is determined to be present in a well, the transmembrane protein expressed by the transfected cells in the well is identified as a transmembrane protein that binds PGRN.

In one embodiment of the high-throughput screening method, the host cells are selected from the group consisting of human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

In a further embodiment of the high-throughput screening, the fixing agent is selected from glutaraldehyde, methanol and paraformaldehyde.

In a yet further embodiment of the high-throughput screening method, the detectable tag is selected from the group consisting of green fluorescent protein (GFP), myc, myc-pyruvate kinase (myc-PK), His6, maltose binding protein (MBP), human influenza virus hemagglutinin (HA) tag, flag tag (FLAG), and glutathione-S-transferase (GST) tag.

In a yet further embodiment of the high-throughput screening method, the target protein is a FLAG tagged PGRN. In one aspect, step h) comprises h1) adding an anti-FLAG antibody that is covalently attached to a fluorescent dye into the wells containing the transfected cells to immunostain the FLAG tagged PGRN, if present, and h2) detecting the fluorescent dye using a fluorometer or an imager in a high-throughput manner. In another aspect, the fluorescent dye is cyanine dye.

In a yet further embodiment of the high-throughput screening method, the target protein is an HA tagged PGRN. In one aspect, step h) comprises h1) exposing the cells to an anti-HA antibody that is covalently attached to a fluorescent dye to immunostain the HA tagged PGRN, if present, and h2) detecting the fluorescent dye using a fluorometer or an imager. In another aspect, the fluorescent dye is Dylight 650.

In a yet further embodiment of the high-throughput screening method, between steps g) and h), the method further comprises permeabilizing the fixed cells with a permeabilizing agent and washing the permeabilized cells.

In a yet further embodiment of the high-throughput screening method, the permeabilizing agent is selected from the group consisting of Saponin, Triton X-100, lysolecithin, n-Octyl-B-D-glucopyranoside, and Tween 20.

Further provided herein is a method for identifying intracellular proteins that bind PGRN, comprising: a) introducing into host cells an expression vector encoding a heterologous protein, thereby producing transfected cells; b) culturing the transfected cells under a condition to allow expression of the heterologous protein; c) permeabilizing the transfected cells with a permeabilizing agent; d) contacting the permeabilized cells with a target protein comprising PGRN attached to a detectable tag; e) fixing the transfected cells with a fixing agent after contact with the target protein; f) washing the fixed cells; and g) after washing the fixed cells, determining the presence of the detectable tag on the washed cells, wherein if the detectable tag is determined to be present, the heterologous protein expressed by the cells is identified as an intracellular protein that binds PGRN.

In one embodiment of the method, the host cells are selected from the group consisting of human embryonic kidney 293T (HEK293T) cell, HEK293F cells, HeLa cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

In a further embodiment of the method, the detectable tag is selected from the group consisting of green fluorescent protein (GFP), myc, myc-pyruvate kinase (myc-PK), His6, maltose binding protein (MBP), human influenza hemagglutinin (HA) tag, flag tag (FLAG), and glutathione-S-transferase (GST) tag.

In a yet further embodiment of the method, the target protein is a FLAG tagged PGRN. In one aspect, step g) comprises g1) exposing the cells to an anti-FLAG antibody that is covalently attached to a fluorescent dye to immunostain the FLAG tagged PGRN, if present, and g2) detecting the fluorescent dye using a fluorometer or an imager. In another aspect, the fluorescent dye is cyanine dye.

In a yet further embodiment of the high-throughput screening method, the target protein is an HA tagged PGRN. In one aspect, step g) comprises g1) exposing the cells to an anti-HA antibody that is covalently attached to a fluorescent dye to immunostain the HA tagged PGRN, if present, and g2) detecting the fluorescent dye using a fluorometer or an imager. In another aspect, the fluorescent dye is Dylight 650.

In a yet further embodiment of the method, between steps b) and c), the method further comprises, fixing the cultured cells with the fixing agent and washing the fixed cells.

In a yet further embodiment of the method, the fixing agent is selected from glutaraldehyde, methanol and paraformaldehyde.

In a yet further embodiment of the method, between steps f) and g), the method further comprises permeabilizing the fixed cells with a permeabilizing agent and washing the permeabilized cells.

In a yet further embodiment of the method, the permeabilizing agent is selected from the group consisting of Saponin, Triton X-100, lysolecithin, n-Octyl-B-D-glucopyranoside, and Tween 20.

Yet further provided herein is a high-throughput screening method for identifying intracellular proteins that bind PGRN, comprising: a) providing a cDNA collection comprising a plurality of expression vectors, each expression vector encoding a distinct heterologous protein; b) providing a plate comprising a plurality of wells, wherein one or more wells thereof each contains host cells; c) into each of the one or more wells, introducing a distinct expression vector from said collection, thereby producing distinct transfected cells in each well; d) culturing the transfected cells under a condition to allow expression of a distinct heterologous protein in each well; e) permeabilizing the transfected cells with a permeabilizing agent; f) contacting the permeabilized cells in each of the one or more wells with a target protein comprising PGRN attached to a detectable tag; g) fixing the transfected cells with a fixing agent after contact with the target protein; h) washing the fixed cells; and i) after washing the fixed cells, determining the presence of the detectable tag in each of the one or more wells, wherein if the detectable tag is determined to be present in a well, the heterologous protein expressed by the transfected cells in the well is identified as a intracellular protein that binds PGRN.

In one embodiment of the high-throughput screening method, the host cells are selected from the group consisting of human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

In a further embodiment of the high-throughput screening method, the detectable tag is selected from the group consisting of green fluorescent protein (GFP), myc, myc-pyruvate kinase (myc-PK), His6, maltose binding protein (MBP), human influenza hemagglutinin (HA) tag, flag tag (FLAG), and glutathione-S-transferase (GST) tag.

In a yet further embodiment of the high-throughput screening method, the target protein is a FLAG tagged PGRN. In one aspect, step i) comprises i1) adding an anti-FLAG antibody that is covalently attached to a fluorescent dye into the wells containing the transfected cells to immunostain the FLAG tagged PGRN, if present, and i2) detecting the fluorescent dye using a fluorometer or an imager in a high-throughput manner. In another aspect, the fluorescent dye is cyanine dye.

In a yet further embodiment of the high-throughput screening method, the target protein is an HA tagged PGRN. In one aspect, step i) comprises i1) exposing the cells to an anti-HA antibody that is covalently attached to a fluorescent dye to immunostain the HA tagged PGRN, if present, and i2) detecting the fluorescent dye using a fluorometer or an imager. In another aspect, the fluorescent dye is Dylight 650.

In a yet further embodiment of the high-throughput screening method, between steps d) and e), the method further comprises, fixing the cultured cells with the fixing agent and washing the fixed cells.

In a yet further embodiment of the high-throughput screening method, the fixing agent is selected from glutaraldehyde, methanol and paraformaldehyde.

In a yet further embodiment of the high-throughput screening method, between steps h) and i), the method further comprises permeabilizing the fixed cells with a permeabilizing agent and washing the permeabilized cells.

In a yet further embodiment of the high-throughput screening method, the permeabilizing agent is selected from the group consisting of Saponin, Triton X-100, lysolecithin, n-Octyl-B-D-glucopyranoside, and Tween 20.

### BRIEF DESCRIPTION OF THE DRAWINGS

FIG. 1 is a schematic depicting a screening strategy for detection of intracellular proteins that bind PGRN.

FIG. 2 shows the Cy3 intensity of cells transfected with pcDNA3 vector (negative control), cells transfected with an expression vector carrying coding sequence of sortilin (positive control), and cells transfected with an expression plasmid carrying the coding sequence of a hit intracellular protein.

FIGS. 3A and 3C shows the Cy3 intensity or Dylight 650 intensity of the transfected cells (negative control, positive control, and Hit #1) that were exposed to FLAG tagged PGRN or HA tagged PGRN, followed by immunostaining with anti-Flag M2-Cy3 antibody or Dylight 650 anti-HA antibody, respectively. FIG. 3B shows the Cy3 signal and Dylight 650 signal for cells transfected with Hit #1 plasmid but not exposed to tagged PGRN prior to immunostaining.

FIG. 4 shows the Cy3 intensity or Dylight 650 intensity of the transfected cells (negative control, positive control, and Hit #1) that were not permeabilized prior to exposure to FLAG tagged PGRN or HA tagged PGRN and immunostaining.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/ml includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2 111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Identification of Transmembrane Proteins that Bind PGRN

In one aspect, the invention disclosed herein are methods for screening for transmembrane proteins that bind to progranulin (PGRN) in a high throughput (HTP) manner.

In particular, a target protein comprised of PGRN fused with a detectable tag, a cDNA library or collection, and host cells are utilized in the screening methods.

PGRN is a cysteine-rich, secreted protein encoded by the GRN gene (NCBI Accession #NM_002087.3). In forming the target protein, PGRN is fused with a detectable tag peptide. Such tag peptides are well known in the art and include, without limitation, green fluorescent protein (GFP), myc, myc-pyruvate kinase (myc-PK), polyhistidine (His6) tag, maltose binding protein (MBP), human influenza virus hemagglutinin (HA) tag (YPYDVPDYA, SEQ ID NO: 1), flag tag (FLAG) (DYKDDDDK, SEQ ID NO: 2), and glutathione-S-transferase (GST) tag. However, the invention should in no way be construed to be limited to the above-listed tags. Rather, any peptide or polypeptide which may function in a manner substantially similar to these tags should be construed to be included in the present invention.

The cDNA library comprises a plurality of expression vectors each encoding a distinct transmembrane protein. In one embodiment, each of the expression vectors encodes a human transmembrane protein. The term “vector” refers to a nucleic acid molecule capable of transporting another nucleic acid to which it has been linked. One type of vector is a “plasmid”, which refers to a circular double-stranded DNA loop into which additional DNA segments can be inserted. Another type of vector is a viral vector wherein additional DNA segments can be inserted. Expression vectors are those vectors capable of directing the expression of genes to which they are operably linked. The expression vectors used herein comprise a nucleic acid encoding a protein sequence in a form suitable for expression of the nucleic acid in a host cell. Thus, the expression vectors can include one or more regulatory sequences, such as a promoter, selected on the basis of the host cells to be used for expression, operably linked to the nucleic acid sequence to be expressed. When used in reference to a expression vector, “operably linked” is intended to mean that the nucleotide sequence of interest is linked to the regulatory sequence(s) in a manner allowing for expression of the nucleotide sequence (e.g., in an in vitro transcription/translation system or in a host cell when the vector is introduced into the host cell). It will be appreciated by those of ordinary skill in the art that the design of the expression vector can depend on such factors as the choice of the host cell to be transformed and the level of expression of protein desired as well as the intended use of the vector. Exemplary cDNA libraries suitable herein may include, without limitation, Ultimate® ORF Clones from ThermoFisher, Human cDNA Clone Sets for Pathway Study or HTP Screening from OriGene, etc.

Any suitable host cells may be used herein. In one embodiment, the host cells used herein are mammalian cells. Suitable mammalian cells may be selected from human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

The high-throughput screening method for identifying transmembrane proteins that bind progranulin (PGRN) uses multi-well plates, such as 96-well, 384-well, or 1536-well plates, and wherein the host cells in culture media are placed in at least one well of the plate. In a high-throughput manner, expression vectors from the cDNA library are introduced into each of the wells containing host cells and the multi-well plate is maintained under suitable culturing conditions to allow transfection and expression of the transmembrane proteins encoded by the expression vectors. The culture media used herein are not particularly limited. Exemplary culture media include, without limitation, Dulbecco's Modified Eagle Medium (DMEM), Roswell Park Memorial Institute 1640 Medium (RPMI 1640), Ham's F-12 Nutrient Mixture (F-12), Dulbecco's Modified Eagle Medium: Nutrient Mixture F-12 (DMEM/F-12) and Minimum Essential Medium (MEM). Suitable culturing conditions may be with 5% CO₂ and at about 30-37° C. for 1-3 days.

After the culturing media is removed, in a high-throughput manner, the target protein (i.e., PGRN fused with detectable tag) is dispensed into each of the wells containing the transfected host cells and the multi-well plate is maintained under a suitable condition to allow potential bond between the transmembrane proteins and the target protein. In one embodiment, the transfected cells are maintained in contact with the target protein at about 20-37° C. for 1-3 hours. Then, fixing agent is added into each well of the multi-well plate to fix the transfected cells followed by washing with washing buffer to remove the un-bond target protein. Examples of suitable fixing agents include glutaraldehyde, methanol and paraformaldehyde. The range of concentrations of these fixatives is generally 0.01 to 8% for paraformaldehyde, and 0.001 to 2% for glutaraldehyde. Combinations of these two reagents may also be employed. Preferred concentrations of paraformaldehyde are 0.1 to 6% with the most preferred concentrations of 1 to 5%. Preferred concentrations of glutaraldehyde are 0.01 to 1% with the most preferred concentrations of 0.05 to 0.5%. The particular concentrations of fixatives are adjusted to optimally fix a particular cell type. Examples of suitable washing buffer include HEPES Balanced Salt Solution (HBSS), Phosphate-Buffered Saline (PBS), Tris-Buffered Saline (TBS), etc.

Finally, the plate is screened for the presence of the detectable tag in each well, wherein if the detectable tag is determined to be present in a well, the transmembrane protein expressed by the transfected cells contained in the well is identified as a transmembrane protein binds PGRN. Preferably the screening for the detectable tag is an automated process.

Optionally, prior to the final screening for the detectable tag, the cells in each well are permeabilized with a permeabilization agent, followed by washing with washing buffer. Permeabilization agents generally are detergent or detergent-like compounds and may include, without limitation, Saponin, Triton X-100, lysolecithin, n-Octyl-B-D-glucopyranoside, and Tween 20.

Starting from placing host cells in the one or more wells until screening for the presence of the detectable tag, each step of the process may be performed in a high-throughput manner, such as by a robotic arm.

In one embodiment, the detectable tag is FLAG tag and the target protein is a N-terminal FLAG tagged PGRN. The final screening includes, adding an anti-FLAG antibody that is covalently attached to a fluorescent dye (such as a cyanine dye, e.g., Cy3 or Cy5) into the wells containing the transfected cells to immunostain the FLAG tagged PGRN, if present, and detecting the fluorescent dye using, for example, fluorometers or imagers.

N-terminal FLAG tagged PGRN:
SEQ ID NO: 3
ATGTGGACTCTGGTCTCATGGGTGGCTCTGACTGCTGGACTGGTG
GCTGGAACCGACTACAAGGACGACGACGACAAACTCGCTGCCCTG
ACGGCCAGTTCTGCGACAAATGGCCCACAACACTGAGCAGGCATC
TGGGTGGCCCCTGCCAGGTTGATGCCCACTGCTCTGCCGGCCACT
CCTGCATCTTTACCGTCTCAGGGACTTCCAGTTGCTGCCCCTTCC
CAGAGGCCGTGGCATGCGGGGATGGCCATCACTGCTGCCCACGGG
GCTTCCACTGCAGTGCAGACGGGCGATCCTGCTTCCAAAGATCAG
GTAACAACTCCGTGGGTGCCATCCAGTGCCCTGATAGTCAGTTCG
AATGCCCGGACTTCTCCACGTGCTGTGTTATGGTCGATGGCTCCT
GGGGGTGCTGCCCCATGCCCCAGGCTTCCTGCTGTGAAGACAGGG
TGCACTGCTGTCCGCACGGTGCCTTCTGCGACCTGGTTCACACCC
GCTGCATCACACCCACGGGCACCCACCCCCTGGCAAAGAAGCTCC
CTGCCCAGAGGACTAACAGGGCAGTGGCCTTGTCCAGCTCGGTCA
TGTGTCCGGACGCACGGTCCCGGTGCCCTGATGGTTCTACCTGCT
GTGAGCTGCCCAGTGGGAAGTATGGCTGCTGCCCAATGCCCAACG
CCACCTGCTGCTCCGATCACCTGCACTGCTGCCCCCAAGACACTG
TGTGTGACCTGATCCAGAGTAAGTGCCTCTCCAAGGAGAACGCTA
CCACGGACCTCCTCACTAAGCTGCCTGCGCACACAGTGGGGGATG
TGAAATGTGACATGGAGGTGAGCTGCCCAGATGGCTATACCTGCT
GCCGTCTACAGTCGGGGGCCTGGGGCTGCTGCCCTTTTACCCAGG
CTGTGTGCTGTGAGGACCACATACACTGCTGTCCCGCGGGGTTTA
CGTGTGACACGCAGAAGGGTACCTGTGAACAGGGGCCCCACCAGG
TGCCCTGGATGGAGAAGGCCCCAGCTCACCTCAGCCTGCCAGACC
CACAAGCCTTGAAGAGAGATGTCCCCTGTGATAATGTCAGCAGCT
GTCCCTCCTCCGATACCTGCTGCCAACTCACGTCTGGGGAGTGGG
GCTGCTGTCCAATCCCAGAGGCTGTCTGCTGCTCGGACCACCAGC
ACTGCTGCCCCCAGGGCTACACGTGTGTAGCTGAGGGGCAGTGTC
AGCGAGGAAGCGAGATCGTGGCTGGACTGGAGAAGATGCCTGCCC
GCCGGGCTTCCTTATCCCACCCCAGAGACATCGGCTGTGACCAGC
ACACCAGCTGCCCGGTGGGGCAGACCTGCTGCCCGAGCCTGGGTG
GGAGCTGGGCCTGCTGCCAGTTGCCCCATGCTGTGTGCTGCGAGG
ATCGCCAGCACTGCTGCCCGGCTGGCTACACCTGCAACGTGAAGG
CTCGATCCTGCGAGAAGGAAGTGGTCTCTGCCCAGCCTGCCACCT
TCCTGGCCCGTAGCCCTCACGTGGGTGTGAAGGACGTGGAGTGTG
GGGAAGGACACTTCTGCCATGATAACCAGACCTGCTGCCGAGACA
ACCGACAGGGCTGGGCCTGCTGTCCCTACCGCCAGGGCGTCTGTT
GTGCTGATCGGCGCCACTGCTGTCCTGCTGGCTTCCGCTGCGCAG
CCAGGGGTACCAAGTGTTTGCGCAGGGAGGCCCCGCGCTGGGACG
CCCCTTTGAGGGACCCAGCCTTGAGACAGCTGCTGTGAGGGACAG
TACTGAAGACTCTGCAGCCCTCGGGACCCCACTCGGAGGGTGCCC
TCTGCTCA
SEQ ID NO: 4
MWTLVSWVALTAGLVAGTDYKDDDDKRCPDGQFCPVACCLDPGGA
SYSCCRPLLDKWPTTLSRHLGGPCQVDAHCSAGHSCIFTVSGTSS
CCPFPEAVACGDGHHCCPRGFHCSADGRSCFQRSGNNSVGAIQCP
DSQFECPDFSTCCVMVDGSWGCCPMPQASCCEDRVHCCPHGAFCD
LVHTRCITPTGTHPLAKKLPAQRTNRAVALSSSVMCPDARSRCPD
GSTCCELPSGKYGCCPMPNATCCSDHLHCCPQDTVCDLIQSKCLS
KENATTDLLTKLPAHTVGDVKCDMEVSCPDGYTCCRLQSGAWGCC
PFTQAVCCEDHIHCCPAGFTCDTQKGTCEQGPHQVPWMEKAPAHL
SLPDPQALKRDVPCDNVSSCPSSDTCCQLTSGEWGCCPIPEAVCC
SDHQHCCPQGYTCVAEGQCQRGSEIVAGLEKMPARRASLSHPRDI
GCDQHTSCPVGQTCCPSLGGSWACCQLPHAVCCEDRQHCCPAGYT
CNVKARSCEKEVVSAQPATFLARSPHVGVKDVECGEGHFCHDNQT
CCRDNRQGWACCPYRQGVCCADRRHCCPAGFRCAARGTKCLRREA
PRWDAPLRDPALRQLL

In one embodiment, the detectable tag is HA tag and the target protein is a N-terminal HA tagged PGRN. The final screening includes, adding an anti-HA antibody that is covalently attached to a dye (such as Dylight 650® anti-HA antibody) into the wells containing the transfected cells to immunostain the HA tagged PGRN, if present, and detecting the dye.

N-terminal HA tagged PGRN:
SEQ ID NO: 5
ATGTGGACTCTGGTCTCATGGGTGGCTCTGACTGCTGGACTGGTG
GCTGGAACCTACCCATACGATGTTCCAGATTACGCTCGCTGCCCT
GACGGCCAGTTCTGCGACAAATGGCCCACAACACTGAGCAGGCAT
CTGGGTGGCCCCTGCCAGGTTGATGCCCACTGCTCTGCCGGCCAC
TCCTGCATCTTTACCGTCTCAGGGACTTCCAGTTGCTGCCCCTTC
CCAGAGGCCGTGGCATGCGGGGATGGCCATCACTGCTGCCCACGG
GGCTTCCACTGCAGTGCAGACGGGCGATCCTGCTTCCAAAGATCA
GGTAACAACTCCGTGGGTGCCATCCAGTGCCCTGATAGTCAGTTC
GAATGCCCGGACTTCTCCACGTGCTGTGTTATGGTCGATGGCTCC
TGGGGGTGCTGCCCCATGCCCCAGGCTTCCTGCTGTGAAGACAGG
GTGCACTGCTGTCCGCACGGTGCCTTCTGCGACCTGGTTCACACC
CGCTGCATCACACCCACGGGCACCCACCCCCTGGCAAAGAAGCTC
CCTGCCCAGAGGACTAACAGGGCAGTGGCCTTGTCCAGCTCGGTC
ATGTGTCCGGACGCACGGTCCCGGTGCCCTGATGGTTCTACCTGC
TGTGAGCTGCCCAGTGGGAAGTATGGCTGCTGCCCAATGCCCAAC
GCCACCTGCTGCTCCGATCACCTGCACTGCTGCCCCCAAGACACT
GTGTGTGACCTGATCCAGAGTAAGTGCCTCTCCAAGGAGAACGCT
ACCACGGACCTCCTCACTAAGCTGCCTGCGCACACAGTGGGGGAT
GTGAAATGTGACATGGAGGTGAGCTGCCCAGATGGCTATACCTGC
TGCCGTCTACAGTCGGGGGCCTGGGGCTGCTGCCCTTTTACCCAG
GCTGTGTGCTGTGAGGACCACATACACTGCTGTCCCGCGGGGTTT
ACGTGTGACACGCAGAAGGGTACCTGTGAACAGGGGCCCCACCAG
GTGCCCTGGATGGAGAAGGCCCCAGCTCACCTCAGCCTGCCAGAC
CCACAAGCCTTGAAGAGAGATGTCCCCTGTGATAATGTCAGCAGC
TGTCCCTCCTCCGATACCTGCTGCCAACTCACGTCTGGGGAGTGG
GGCTGCTGTCCAATCCCAGAGGCTGTCTGCTGCTCGGACCACCAG
CACTGCTGCCCCCAGGGCTACACGTGTGTAGCTGAGGGGCAGTGT
CAGCGAGGAAGCGAGATCGTGGCTGGACTGGAGAAGATGCCTGCC
CGCCGGGCTTCCTTATCCCACCCCAGAGACATCGGCTGTGACCAG
CACACCAGCTGCCCGGTGGGGCAGACCTGCTGCCCGAGCCTGGGT
GGGAGCTGGGCCTGCTGCCAGTTGCCCCATGCTGTGTGCTGCGAG
GATCGCCAGCACTGCTGCCCGGCTGGCTACACCTGCAACGTGAAG
GCTCGATCCTGCGAGAAGGAAGTGGTCTCTGCCCAGCCTGCCACC
TTCCTGGCCCGTAGCCCTCACGTGGGTGTGAAGGACGTGGAGTGT
GGGGAAGGACACTTCTGCCATGATAACCAGACCTGCTGCCGAGAC
AACCGACAGGGCTGGGCCTGCTGTCCCTACCGCCAGGGCGTCTGT
TGTGCTGATCGGCGCCACTGCTGTCCTGCTGGCTTCCGCTGCGCA
GCCAGGGGTACCAAGTGTTTGCGCAGGGAGGCCCCGCGCTGGGAC
GCCCCTTTGAGGGACCCAGCCTTGAGACAGCTGCTGTGAGGGACA
GTACTGAAGACTCTGCAGCCCTCGGGACCCCACTCGGAGGGTGCC
CTCTGCTCA
SEQ ID NO: 6
MWTLVSWVALTAGLVAGTYPYDVPDYARCPDGQFCPVACCLDPGG
ASYSCCRPLLDKWPTTLSRHLGGPCQVDAHCSAGHSCIFTVSGTS
SCCPFPEAVACGDGHHCCPRGFHCSADGRSCFQRSGNNSVGAIQC
PDSQFECPDFSTCCVMVDGSWGCCPMPQASCCEDRVHCCPHGAFC
DLVHTRCITPTGTHPLAKKLPAQRTNRAVALSSSVMCPDARSRCP
DGSTCCELPSGKYGCCPMPNATCCSDHLHCCPQDTVCDLIQSKCL
SKENATTDLLTKLPAHTVGDVKCDMEVSCPDGYTCCRLQSGAWGC
CPFTQAVCCEDHIHCCPAGFTCDTQKGTCEQGPHQVPWMEKAPAH
LSLPDPQALKRDVPCDNVSSCPSSDTCCQLTSGEWGCCPIPEAVC
CSDHQHCCPQGYTCVAEGQCQRGSEIVAGLEKMPARRASLSHPRD
IGCDQHTSCPVGQTCCPSLGGSWACCQLPHAVCCEDRQHCCPAGY
TCNVKARSCEKEVVSAQPATFLARSPHVGVKDVECGEGHFCHDNQ
TCCRDNRQGWACCPYRQGVCCADRRHCCPAGFRCAARGTKCLRRE
APRWDAPLRDPALRQL

Preferably, a high-content throughput imager is used to detect the tagged PGRN.

Identification of Intracellular Proteins that Bind PGRN

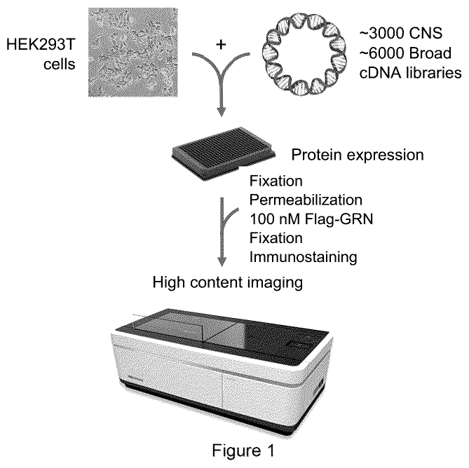

In another aspect, the invention disclosed herein are methods for screening for intracellular proteins that bind to progranulin (PGRN). A schematic outline of the method is shown in FIG. 1.

In this method the target protein comprised of PGRN fused with a detectable tag (as described above), a cDNA library or collection comprising a plurality of expression vectors each encoding a distinct protein (preferably human proteins, more preferably human intracellular proteins), and host cells (as described above), are utilized.

The method includes introducing an expression vector from the cDNA library into a host cell and maintaining the expression vector and the host cell in a suitable culture media under suitable culturing conditions to allow transfection and expression of the protein encoded by the expression vector. Suitable culturing conditions are the same as those described above.

After removing the culture media, the transfected cells are permeabilized with a permeabilizing agent (as described above) followed by washing with wash buffer. The permeabilizing agent can sufficiently disrupt the cellular membrane of the cells to permit target proteins to enter the cell. The permeabilized cells are then brought into contact with the target protein and maintained under a suitable condition (as described above) to allow potential bond between the intracellular proteins and the target protein. Optionally, prior to permeabilization, the transfected cells are fixed with a fixing agent followed by washing with washing buffer.

After contacting the target protein, the cells are fixed with a fixing agent (as described above) and washed with washing buffer to remove the un-bond target protein.

Finally, the presence of the detectable tag is determined, wherein if the detectable tag is determined to be present, the protein encoded by the expression vector is identified as an intracellular protein binds PGRN.

Optionally, prior to detecting the detectable tag, the fixed cells are again permeabilized with a permeabilization agent, followed by washing with washing buffer.

In one embodiment, the detectable tag is FLAG tag and the target protein is a N-terminal FLAG tagged PGRN. To determine the presence of the FLAG tag, an anti-FLAG antibody that is covalently attached to a fluorescent dye (such as a cyanine dye, e.g., Cy3 or Cy5) is added to immunostain the FLAG tagged PGRN, if present, and an imager or fluorometer is used to detect the presence of the fluorescent dye on the cells.

In one embodiment, the detectable tag is HA tag and the target protein is a N-terminal HA tagged PGRN. The final screening includes, adding an anti-HA antibody that is covalently attached to a dye (such as Dylight 650® anti-HA antibody) into the wells containing the transfected cells to immunostain the HA tagged PGRN, if present, and detecting the dye.

In one embodiment, the screening for intracellular proteins that bind to progranulin (PGRN) is conducted in a high-throughput manner. In the high-throughput process, multi-well plates, such as 96-well, 384-well, or 1536-well plates, are used. Host cells in a suitable culture media are placed in one or more wells of the plate. And starting from placing host cells in the one or more wells until screening for the presence of the detectable tag, each step of the process is performed in a high-throughput manner, such as by a robotic arm.

### EXAMPLE

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Identification of Cell Surface Receptor for Progranulin

In this study, a cDNA collection containing over 4,000 transfection-ready cDNA plasmids for both human and mouse proteins with a transmembrane domain (obtained from OriGene) was used. Human open reading frames (ORFs) are located on pCMV6-AC pCMV6-XL4, pCMV6-XL5, pCMV6-XL6, or pCMV6-NEO backbones and mouse ORFs are located on a pCMV6-Kan/Neo skeleton. The cDNA plasmids were transfected individually into HEK293T cells (ATCC) in 384-well poly-D-lysine coated plates (Perkin Elmer) using Fugene 6 (Promega). Each well was seeded with 10,000 cells. Using Fugene 6 according to the manufacturer's instructions, each well was transfected with 60 ng of plasmid DNA, 0.3 ng of which was GFP plasmid (green fluorescent protein (GFP) gene expressed on pcDNA3.1) as a transfection efficiency control. The cells were incubated for 1 day at 37° C., after which they were moved to 30° C. and incubated for 2 days. Three (3) days after transfection, cells in each well were incubated with 100 nM N-terminal flag tagged PGRN at room temperature for 2 hours. Following PGRN incubation, the media was removed and the cells were fixed with 4% paraformaldehyde for 40 minutes at room temperature. Afterwards, the cells were washed with HEPES Balanced Salt Solution (HBSS) 3 times, and permeabilized with 0.3% Triton X-100 for 30 minutes at room temperature. After permeabilization, cells were washed 3 times again, and then immunostained with 400 ng/mL of Anti-Flag M2-Cy3 antibody (Sigma) at 4° C. overnight. The following day, the cells were washed 3 times again and then stained with NucBlue Live ReadyProbes Reagent (Invitrogen) according to manufacturer's instructions. Subsequently, the plate was imaged using the Opera Phenix high-content throughput imaging machine (Perkin Elmer).

### Identification of Intracellular Binding Partner for Progranulin

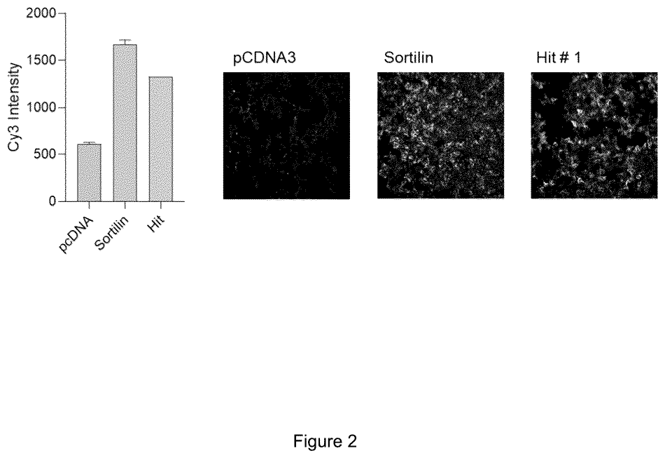

Two separate cDNA collections of around 9,000 total plasmids were screened. The first cDNA collection includes about 3000 transfection-ready cDNA plasmids for both human and mouse proteins expressed in the central nervous system (CNS). Human ORFs are similar to the above, located on pCMV6-AC pCMV6-XL4, pCMV6-XL5, pCMV6-XL6, or pCMV6-NEO backbones. Mouse ORFs in this collection are located on a pCMV6-Entry backbone. Some of the proteins in this collection were tagged with a Myc-DDK tag, in which case HA-tagged PGRN was used for screening. The second cDNA collection containing about 6000 transfection-ready cDNA plasmids selected from the hORFeome V8.1 library (obtained from the Broad Institute). The plasmids were transfected individually into HEK293T cells (ATCC) in 384-well poly-D-lysine coated plates (Perkin Elmer) using Fugene 6 (Promega). Each well was seeded with 10,000 cells. Using Fugene 6 according to the manufacturer's instructions, each well was transfected with 60 ng of plasmid DNA, 3 ng of which was GFP plasmid as a transfection control. The cells were incubated for 1 day at 37° C., after which they were moved to 30° C. and incubated for 2 days. Three (3) days after transfection the media was removed and the cells were fixed with 4% paraformaldehyde for 40 minutes at room temperature. Afterwards, the cells were washed with HBSS 3 times, and permeabilized with 0.3% Triton X-100 for 30 minutes at room temperature. After permeabilization, cells were washed 3 times again, and incubated in Dulbecco's Modified Eagle Medium (DMEM) containing 100 nM N-terminal flag tagged PGRN for 2 hours at room temperature. After incubation with PGRN, the DMEM was removed and the cells were fixed again with 4% paraformaldehyde for 40 minutes at room temperature. After the 2nd fixation, the cells were washed again with HBSS for 3 times and then immunostained with 400 ng/ml of Anti-Flag M2-Cy3 antibody (Sigma) in 4° C. overnight. The following day, the cells were washed 3 times again and then stained with NucBlue Live ReadyProbes Reagent (Invitrogen) according to manufacturer's instructions. Subsequently, the plate was imaged using the Opera Phenix high-content throughput imaging machine (Perkin Elmer). FIG. 2 show the Cy3 intensity for cells transfected with pcDNA3 vector (negative control) and cells transfected with an expression vector carrying the coding sequence of sortilin (positive control). Also shown in FIG. 2 is the Cy3 intensity for cells transfected with one of the 9000 expression plasmids, which is comparable to the positive control. Thus, the intracellular protein encoded by the expression plasmid was identified as Hit #1.

### Confirmation of Hit #1

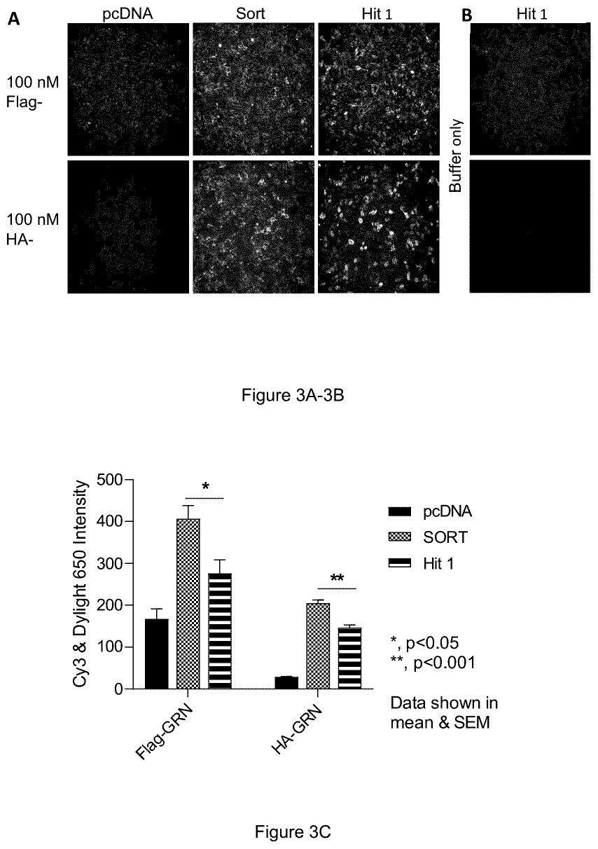

The plasmid for Hit #1, along with pcDNA3 vector (negative control) and an expression vector carrying the coding sequence of sortilin (positive control) were individually transfected into HEK293 cells. Following the same process described under “Identification of Intracellular Binding Partner for Progranulin”, the transfected cells were exposed to N-terminal FLAG tagged PGRN or N-terminal HA tagged PGRN, followed by immunostaining with anti-Flag M2-Cy3 antibody and Dylight 650 anti-HA antibody, respectively. As shown in FIGS. 3A and 3C, the Cy3 and Dylight intensity for cells transfected with Hit #1 plasmid was comparable to that for the positive control. In addition, the cells transfected with Hit #1 plasmid was also immunostained with anti-Flag M2-Cy3 antibody and Dylight 650 anti-HA antibody without prior exposure to the FLAG tagged PGRN or HA tagged PGRN. And as shown in FIG. 3B, in both cases, the fluorescent signal was the same as the negative control, confirming that the identification of Hit #1 was not due to non-specific antibody binding to the cells transfected with the Hit #1 plasmid.

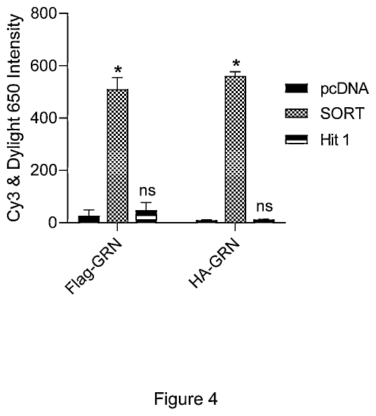

Further, following the same process described under “Identification of Cell Surface Receptor for Progranulin”, HEK293 cells transfected with pcDNA3 vector (negative control), expression vector carrying the coding sequence of sortilin (positive control), and Hit #1 plasmid were exposed to FLAG tagged PGRN or HA tagged PGRN without being permeabilized, followed by immunostaining with anti-Flag M2-Cy3 antibody and Dylight 650 anti-HA antibody, respectively. As shown, in FIG. 4, the fluorescent signal intensity for Hit #1 was the same as the negative control, confirming that Hit #1 is not a surface receptor for PGRN.

What is claimed is:

1. A high-throughput screening method for identifying transmembrane proteins that bind progranulin (PGRN), comprising:
a) providing a cDNA collection comprising a plurality of expression vectors, each expression vector encoding a distinct transmembrane protein;
b) providing a plate comprising a plurality of wells, wherein one or more wells thereof each contains host cells;
c) into each of the one or more wells, introducing a distinct expression vector from said collection, thereby producing distinct transfected cells in each well;
d) culturing the transfected cells under a condition to allow expression of a distinct transmembrane protein in each well;
e) contacting the transfected cells in each of the one or more wells with a target protein comprising PGRN attached to a detectable tag;
f) fixing the transfected cells with a fixing agent after contact with the target protein;
g) washing the fixed cells; and
h) after washing the fixed cells, determining the presence of the detectable tag in each of the one or more wells, wherein if the detectable tag is determined to be present in a well, the transmembrane protein expressed by the transfected cells in the well is identified as a transmembrane protein that binds PGRN.

2. The high-throughput screening method of claim 1, wherein the host cells are selected from the group consisting of human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

3. The high-throughput screening method of claim 1 or 2, wherein the fixing agent is selected from glutaraldehyde, methanol and paraformaldehyde.

4. The high-throughput screening method of any one of claims 1-3, wherein, the detectable tag is selected from the group consisting of green fluorescent protein (GFP), myc, myc-pyruvate kinase (myc-PK), His6, maltose binding protein (MBP), human influenza virus hemagglutinin (HA) tag, flag tag (FLAG), and glutathione-S-transferase (GST) tag.

5. The high-throughput screening method of claim 4, wherein, the target protein is a FLAG tagged PGRN.

6. The high-throughput screening method of claim 5, wherein, step h) comprises h1) adding an anti-FLAG antibody that is covalently attached to a fluorescent dye into the wells containing the transfected cells to immunostain the FLAG tagged PGRN, if present, and h2) detecting the fluorescent dye using a fluorometer or an imager in a high-throughput manner.

7. The high-throughput screening method of claim 6, wherein the fluorescent dye is cyanine dye.

8. The high-throughput screening method of claim 4, wherein, the target protein is a HA tagged PGRN.

9. The high-throughput screening method of claim 8, wherein, step h) comprises h1) adding an anti-HA antibody that is covalently attached to a fluorescent dye into the wells containing the transfected cells to immunostain the HA tagged PGRN, if present, and h2) detecting the fluorescent dye using a fluorometer or an imager in a high-throughput manner.

10. The high-throughput screening method of claim 6, wherein the fluorescent dye is Dylight 650.

11. The high-throughput screening method of any one of claims 1-10, wherein between steps g) and h), the method further comprises permeabilizing the fixed cells with a permeabilizing agent and washing the permeabilized cells.

12. The high-throughput screening method of claim 11, wherein the permeabilizing agent is selected from the group consisting of Saponin, Triton X-100, lysolecithin, n-Octyl-B-D-glucopyranoside, and Tween 20.

13. A method for identifying intracellular proteins that bind PGRN, comprising:
a) introducing into host cells an expression vector encoding a heterologous protein, thereby producing transfected cells;
b) culturing the transfected cells under a condition to allow expression of the heterologous protein;
c) permeabilizing the transfected cells with a permeabilizing agent;
d) contacting the permeabilized cells with a target protein comprising PGRN attached to a detectable tag;
e) fixing the transfected cells with a fixing agent after contact with the target protein;
f) washing the fixed cells; and
g) after washing the fixed cells, determining the presence of the detectable tag on the washed cells, wherein if the detectable tag is determined to be present, the heterologous protein expressed by the cells is identified as an intracellular protein that binds PGRN.

14. The method of claim 13, wherein the host cells are selected from the group consisting of human embryonic kidney 293T (HEK293T) cell, HEK293F cells, HeLa cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

15. The method of 13 or 14, wherein, the detectable tag is selected from the group consisting of green fluorescent protein (GFP), myc, myc-pyruvate kinase (myc-PK), His6, maltose binding protein (MBP), human influenza virus hemagglutinin (HA) tag, flag tag (FLAG), and glutathione-S-transferase (GST) tag.

16. The method of claim 15, wherein, the target protein is a FLAG tagged PGRN.

17. The method of claim 16, wherein, step g) comprises g1) exposing the cells to an anti-FLAG antibody that is covalently attached to a fluorescent dye to immunostain the FLAG tagged PGRN, if present, and g2) detecting the fluorescent dye using a fluorometer or an imager.

18. The method of claim 17, wherein the fluorescent dye is cyanine dye.

19. The method of claim 15, wherein, the target protein is an HA tagged PGRN.

20. The method of claim 19, wherein, step g) comprises g1) exposing the cells to an anti-HA antibody that is covalently attached to a fluorescent dye to immunostain the HA tagged PGRN, if present, and g2) detecting the fluorescent dye using a fluorometer or an imager.

21. The method of claim 20, wherein the fluorescent dye is Dylight 650.

22. The method of any one of claims 13-21, wherein between steps b) and c), the method further comprises, fixing the cultured cells with the fixing agent and washing the fixed cells.

23. The method of any one of claims 13-22, wherein the fixing agent is selected from glutaraldehyde, methanol and paraformaldehyde.

24. The method of any one of claims 13-23, wherein between steps f) and g), the method further comprises permeabilizing the fixed cells with a permeabilizing agent and washing the permeabilized cells.

25. The method of any one of claims 13-24, wherein the permeabilizing agent is selected from the group consisting of Saponin, Triton X-100, lysolecithin, n-Octyl-B-D-glucopyranoside, and Tween 20.

26. A high-throughput screening method for identifying intracellular proteins that bind PGRN, comprising:
a) providing a cDNA collection comprising a plurality of expression vectors, each expression vector encoding a distinct heterologous protein;
b) providing a plate comprising a plurality of wells, wherein one or more wells thereof each contains host cells;
c) into each of the one or more wells, introducing a distinct expression vector from said collection, thereby producing distinct transfected cells in each well;
d) culturing the transfected cells under a condition to allow expression of a distinct heterologous protein in each well;
e) permeabilizing the transfected cells with a permeabilizing agent;
f) contacting the permeabilized cells in each of the one or more wells with a target protein comprising PGRN attached to a detectable tag;
g) fixing the transfected cells with a fixing agent after contact with the target protein;
h) washing the fixed cells; and
i) after washing the fixed cells, determining the presence of the detectable tag in each of the one or more wells, wherein if the detectable tag is determined to be present in a well, the heterologous protein expressed by the transfected cells in the well is identified as a intracellular protein that binds PGRN.

27. The high-throughput screening method of claim 26, wherein the host cells are selected from the group consisting of human embryonic kidney 293T (HEK293T) cell, HEK293F cells, Hela cells, Chinese hamster ovary (CHO) cells, NIH 3T3 cells, MCF-7 cells, Hep G2 cells, baby hamster kidney (BHK) cells, BV-2 (mouse, C57BL/6, brain, microglial) cells, Neuro-2a (N2a) cells and Cos7 cells.

28. The high-throughput screening method of 26 or 27, wherein, the detectable tag is selected from the group consisting of green fluorescent protein (GFP), myc, myc-pyruvate kinase (myc-PK), His6, maltose binding protein (MBP), influenza virus hemagglutinin tag, flag tag (FLAG), and glutathione-S-transferase (GST) tag.

29. The high-throughput screening method of claim 28, wherein, the target protein is a FLAG tagged PGRN.

30. The high-throughput screening method of claim 29, wherein, step i) comprises i1) adding an anti-FLAG antibody that is covalently attached to a fluorescent dye into the wells containing the transfected cells to immunostain the FLAG tagged PGRN, if present, and i2) detecting the fluorescent dye using a fluorometer or an imager in a high-throughput manner.

31. The high-throughput screening method of claim 30, wherein the fluorescent dye is cyanine dye.

32. The high-throughput screening method of claim 28, wherein, the target protein is an HA tagged PGRN.

33. The high-throughput screening method of claim 32, wherein, step i) comprises i1) adding an anti-HA antibody that is covalently attached to a fluorescent dye into the wells containing the transfected cells to immunostain the HA tagged PGRN, if present, and i2) detecting the fluorescent dye using a fluorometer or an imager in a high-throughput manner.

34. The high-throughput screening method of claim 33, wherein the fluorescent dye is Dylight 650.

35. The high-throughput screening method of any one of claims 26-34, wherein between steps d) and e), the method further comprises, fixing the cultured cells with the fixing agent and washing the fixed cells.

36. The high-throughput screening method of any one of claims 26-35, wherein the fixing agent is selected from glutaraldehyde, methanol and paraformaldehyde.

37. The high-throughput screening method of any one of claims 26-36, wherein between steps h) and i), the method further comprises permeabilizing the fixed cells with a permeabilizing agent and washing the permeabilized cells.

38. The high-throughput screening method of any one of claims 26-37, wherein the permeabilizing agent is selected from the group consisting of Saponin, Triton X-100, lysolecithin, n-Octyl-B-D-glucopyranoside, and Tween 20.
